(19) United States
(12) Patent Application Publication
(10) Pub. No.: US 2006/0068044 A1
(43) Pub. Date: Mar. 30, 2006

(54) Antimicrobial compositions and applications therefore

(76) Inventor: Max Reynolds, Kuraby (AU)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/239,014

(22) Filed: Sep. 30, 2005

Related U.S. Application Data
(60) Provisional application No. 60/688,354, filed on Jun. 8, 2005.
(60) Provisional application No. 60/614,329, filed on Sep. 30, 2004.

(51) Int. Cl. A61K 36/61

(52) U.S. Cl. 424/769

(57) ABSTRACT

The present invention relates to a composition derived from an essential oil obtained from one or more plants of the Melaleuca family in which at least 80% of the monoterpene content of the oil has been removed. The present invention also relates to a number of applications of the composition including antimicrobial, antiviral and therapeutic applications. The present invention in a particular embodiment relates to medical devices to which the composition has been applied and/or absorbed thereon.

### RELATED APPLICATIONS

This application claims the benefit of U.S. provisional Patent application 60/614,329 filed on 30 September and U.S. Provisional Patent Application 60/688,354 filed on 8 Jun. 2005, the specifications of which are incorporated herein by reference.

### FIELD OF THE INVENTION

The present invention relates to a composition derived from an essential oil obtained from one or more plants of the Melaleuca family. In particular, the present invention also relates to therapeutic applications of the composition. The present invention in a particular embodiment relates to medical devices to which the composition has been applied and/or absorbed thereon.

### BACKGROUND OF THE INVENTION

The present invention will be described with particular reference to a composition derived from Melaleuca alternifolia. However, it will be appreciated that compositions of the invention may also be sourced from other Melaleuca species and no limitation is intended thereby. Other suitable sources of compositions of the present invention include but are not limited to M. Bracteata, M. Cricifolia and M. quinquinervia.

Essential oils are complex mixtures of volatile oils produced by plants and are responsible for the odour of many plants. The essential oil, once produced, is either released to the environment or stored in oil cells for later use. Essential oils stored in the wood of plants serves to deter micro-organisms and insects from attack.

Essential oils having antiseptic properties are well known. The essential oil obtained from the steam distillation of the stems and leaves of Melaleuca alternifolia is known as tea tree or Melaleuca oil. It is used widely as a topical antiseptic and in the control of ectoparasites such as fleas and head lice.

Essential oils contain large amounts of terpenes. Terpenes are classified according to the number of units of the basic structure methylbuta-1,3-diene or isoprene, which make up the terpene. Monoterpenes contain two isoprene units and have the chemical formula C₁₀H₁₆. Terpenes may be acyclic such as myrcene and ocimene or cyclic such as limonene. Typically, commercially available Melaleuca oil comprises up to about 50% monoterpenes. Monoterpenes found in melaleuca oil include alpha-pinene, gamma terpinene, alpha terpinene and limonene.

Essential oils typically also contain sesquiterpenes. Sesquiterpenes contain three isoprene units and have the general formula C₁₅H₂₄ and are generally found in much lower quantities than the monoterpenes. For example, Melaleuca oil typically contains about 4 to 8% sesquiterpenes.

Another class of compounds commonly found in essential oils are known as oxygenates. These compounds have an oxygen containing functional group. Examples are aldehydes, phenol alcohols, carboxylic acids, ketones and esters. Terpin-4-ol, having the formula, C₁₀H₁₈O is a major constituent of Melaleuca oil and can constitute up to 40% of the oil. Terpin-4-ol is considered to be the major active constituent of Melaleuca oil. However, other oxygenated products and the monoterpenes are also believed to have some antimicrobial activity.

The composition of commercially available Melaleuca oils is partially regulated by International and Australian Standards. These standards set a minimum terpin-4-ol content of 30% and a maximum 1,8-cineol content of 15%.

Terpenes contain double bonds, which are susceptible to oxidation. It is believed that the capacity to generate activated oxygen intermediates may be responsible for their antimicrobial activity. On the other hand, this susceptibility to oxidation results in instability. Terpenes, particularly monoterpenes, are primarily saturated hydrocarbons, which are vulnerable to oxidation by oxygen in the environment surrounding the monoterpenes. The attack occurs in the region of the C—C double bonds of the terpene molecule. Such instability typically leads to discoloration, odour and premature loss of the proactive sites and also accounts for some of the observed heat sensitivity and chemical reactivity of the essential oils. A further disadvantage is that some of these oxidation products may be irritating or even allergenic.

The present inventor has surprisingly and unexpectedly discovered that a composition derived from Melaleuca essential oil, whereby a major portion of the monoterpene content has been removed, not only exhibits improved stability but also retains and, in some cases, increases its antiseptic and antimicrobial properties. The present inventor has further observed that a preferred composition of the present invention exhibits improved antimicrobial properties when compared with conventional Melaleuca oil. Further still, the present inventor has discovered that whilst conventional Melaleuca oil is suitable only for topical administration in view of its toxicity when ingested, that a preferred composition of the invention may be considered safe for oral administration. Toxic effects, which may be experienced if after ingestion of Melaleuca oil include seizures, coma and respiratory depression.

### SUMMARY OF THE INVENTION

It is therefore an object of the present invention to provide a composition, which may at least partially overcome the above disadvantages or provide the public with a useful or commercial choice. It is also an object of the present invention to provide methods of treatment for conditions associated with, or caused by, infectious agents. A further object is to provide medical devices with biocidal properties.

According to a first broad form of the invention, there is provided a composition derived from an essential oil of a Melaleuca plant, wherein at least about 80% of the monoterpene content of the oil has been removed.

### DESCRIPTION OF THE INVENTION

The essential oil from which the composition is derived may be extracted from any one or more Melaleuca species. Preferably the essential oil is extracted from Melaleuca alternifolia. The essential oil is typically derived by known procedures such as steam distillation.

As discussed above, essential oils derived from Melaleuca species comprise a monoterpene fraction, an oxygenate fraction and a sesquiterpene fraction, although it will be appreciated that different species may contain different relative amounts of each fraction. Of these fractions, the monoterpenes are generally the most volatile and have the lowest molecular weight. Thus, they may be removed by techniques known to those of skill in the art including vacuum low temperature techniques, such as inert gas flushed distillation; molecular weight separation techniques including chromatographic techniques and selective solvent extraction techniques. Preferably, the monoterpenes are removed under reduced pressure and at a temperature that does not exceed 50° C., preferably 40° C. Generally between about 80 and about 99% of the monoterpenes are removed, typically between about 90 and about 99%.

A preferred composition of the invention is derived from the essential oil of Melaleuca alternifolia and typically comprises from between about 40 to about 70%, preferably between about 50 to about 65% most preferably between about 60 to about 65% terpen-4-ol and between about 8 to about 30%, preferably between about 8 to about 25% sesquiterpenes. The sesquiterpene fraction may include aromadendrene, voridiflorene, delta cadinene, globulol and/or viridiflorol.

The composition of the invention may find particular application as an antimicrobial agent.

Thus, according to a further broad form of the invention, there is provided an antimicrobial composition derived from an essential oil of Melaleuca, wherein at least about 80% of the monoterpene content of the oil has been removed.

The antimicrobial composition may find application as an antibacterial, antiprotozoan, antifungal and/or antiviral agent. Typically, the composition is effective against a broad range of micro-organisms including E. Coli, S. aureus, P. aeruginosa, C albicans, S. epidermidis, Penicillium ssp, Cladosporium, A. Niger, A. fumigatus, P. expansum, S. chartarum, Alteraria, Aspergillus, Fusarium, B. subtilis, B. cereus, C. perfringens, K. pneumoniae, L. lactis, M. smegmatis, S. marcescens, S. pyogenes, A. viridans, E. aerogenes, S. liquefaciens, P. vulgaris, S. enteridis, P. mirabilis, S. abaetetuba, L. monocytogenes, N. Gonorrhoeae, Legionella, M. Gordanoae and M. catarrhalis and viruses including coronavirus, rotavirus, adenovirus, herpes simplex, papillovirus, rhinovirus, hepatitis B and A, enterovirus and respiratory viruses such as influenza and parainfluenza virus.

The composition may be used as a disinfectant and/or anti-mould agent for hard surfaces such as those typically found in homes like kitchens, bathrooms, tiles, walls, floors, chrome, glass, smooth vinyl, any plastic, plastified wood, table top, sinks, cooker tops, dishes, sanitary fittings such as sinks, showers, shower curtains, wash basins and the like. Hard-surfaces also include household appliances including but not limited to, refrigerators, freezers, washing machines, automatic dryers, ovens, microwave ovens, dishwashers and surfaces found in hospitals, restaurants, hotels, means of public transport, public bathes and pools, commercial and public laundries and the like are included herein.

The composition may further comprise optional ingredients such as a solvent, surfactant, chelating agent, fragrance, carrier, diluent, one or more other essential oils and the like. Such optional ingredients are known to those of skill in the formulation arts.

The composition of the invention may also be used to control airborne infectious agents. In this case, the composition may be formulated together with a suitable propellant. A preferred propellent is Nitrogen in which droplets of the composition are dispersed.

Infectious agents that may spread via airborne droplets include chickenpox, common cold, diphtheria, haemophilus influenza type b (Hib), influenza, measles, meningitis (bacterial), meningococcal disease, mumps, parvovirus infection (human parvovirus infection, parvovirus B19 infection, slapped cheek, slapped face, erythema infection, fifth disease), Pneumococcal pneumonia, rubella, streptococcal sore throat, tuberculosis and whooping cough (pertussis). The composition of the present invention may be used to control such airborne infectious agents. The compositions may find particular application in otherwise sterile environments such as operating theatres, isolation wards and the like.

According to a further broad form of the invention there is provided an anti-microbial aerosol composition comprising the composition of the first broad form dispersed in a propellent.

According to a further broad form of the invention there is provided a method of controlling airborne infectious agents in an atmospheric environment, the method comprising dispersing an effective amount of the aerosol of the previous embodiment into the environment.

The composition may also find use as a topical anti-microbial and/or anti-parasitic agent for human or animal use. Examples of such applications include hand sanitizers, antiseptic scrubs or washes as well as flea and lice shampoos. The composition may also be used to treat ulcers, cuts, abrasions, wounds, acne, diabetic gangrene and for use in palliative care cancer patients.

The inventor has further surprisingly and unexpectedly discovered that the composition of the present invention may also possess topical analgesic and anti-inflammatory properties.

According to a further broad form of the invention there is provided a topical therapeutic composition comprising a composition of the first broad form.

The topical composition may be in any suitable form including an aerosol, preferably a nitrogen aerosol, a cream, gel or oil. For wound treatment, the aerosol typically has a particle size of between about 5 to about 20, preferably about 10 micron and for an analgesic between about 40 to about 60 micron. Typical carriers for topical administration include water, alcohol, silicone, other essential oils, oils, wax and gels. Typically, for analgesic or anti-inflammatory use, a topical composition comprises between about 1 to about 5% of the inventive composition.

The anti-inflammatory properties of the composition may make it suitable for use in the external treatment of painful muscles, tendons, skin irritations, gout, periodontal disease, gingivitis and the like. The composition may also be suitable for internal use for the treatment of conditions associated with an inflammatory response such as colon, bowel, lung, throat and nose infections.

The topical compositions may also be administered in the form of wound dressings, transdermal patches and the like. Typically wound dressings are impregnated with a composition of the invention at a concentration of active agents of between about 10000 to about 5000 ppm.

A still further use of a composition of the invention is as an antibiotic or anti-viral agent for internal human or animal use. It will be appreciated by those of skill in the art that conventional essential oils containing monoterpenes are considered unsuitable for oral ingestion. Cases of poisoning have been reported after accidental ingestion of commercial Melaleuca oil.

According to a further broad form of the invention, there is provided a pharmaceutical composition for treating an infection in an animal, the composition comprising an effective amount of the composition of the first broad form together with a pharmaceutically effective carrier diluent, excipient and/or adjuvant.

According to a further broad form of the invention, there is provided a method for the treatment or prophylaxis of an infection in an animal, the method comprising administering to the animal an effective amount of the composition of the first broad form of the invention.

The infectious agent includes any agent that may be responsible for, or contribute to, a health condition in an animal. Such infectious agents include bacteria, fungi and viruses and parasitic infectious agents such as malaria, hookworm, tapeworm Giardia and the like.

The pharmaceutical composition may be in any suitable form. Solid form preparations include powders, tablets, dispersible granules, capsules, cachets, suppositories and ointments. Liquid form preparations include solutions, suspensions and emulsions suitable for oral ingestion or injection.

Typical dosage level may be between about 0.001 and about 100 mg/kg body weight per day, preferably between about 0.5 and about 75 mg/kg body weight per day. Typically, the pharmaceutical compositions of this invention will be administered from about 1 to about 5 times per day or alternatively, preferably on a daily basis.

As the skilled artisan will appreciate, lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination and the severity of the condition.

In a particularly preferred form of the invention, the pharmaceutical composition is administered to the lung by the pulmonary route. Such administration may be suitable for the treatment of diseases caused by upper respiratory and lung infections such as tuberculosis, bacterial and viral pneumonia and influenza. The composition may be administered using known facial vaporizers. Preferably, the composition is formulated as an aerosol. Preferably, the aerosol administers particles having a size of up to about 10 microns. Preferably, the aerosol is formulated using nitrogen as a propellant.

The composition of the present invention may also be used in association with medical devices. The device may be treated with the composition in any suitable manner such as by coating, absorption and impregnation, or in the case of thermoplastic materials, co-extruded therewith. The medical devices may be external devices such as catheters, urinary bags and any suitable container that may in use come into contact with an infectious agent. Internal devices may include stents and implants such as breast implants. The surface of a plastic medical device may be pre-treated prior to coating to facilitate absorption on, or adhesion thereto. Such pre-treatment may include surface activation by methods such as corona or plasma treatment, or by sonically disrupting the plastic surface. Alternatively, or in addition to, the composition may be formulated to facilitate attachment to a plastic surface. For example, in the case of a silicone breast implant, the compositions may be formulated in a silicone based carrier or diluent.

The composition of the present invention may also be used as a fabric and/or garment biocide. Suitable fabrics include natural and synthetic fabrics and also fabrics formed from or including cellulosic materials. It is often desirable for sporting or active wear garments to be treated with a biocide, so as to control odour releasing bacteria. Biocidal fabrics find particular application in hospitals or other healthcare situations. The composition of the invention may be used in association with gloves, head covers, gowns, boots, head bands and the like. A particular application is for surgical masks. Typically, surgical masks may be treated by spraying an aerosol onto both sides of the mask.

According to a further broad form of the invention there is provided a fabric to which a composition of the first broad from has been applied.

The composition maybe applied by any suitable means such as spraying, dipping painting or coating.

### EXAMPLES

### Example 1

A composition of the present invention was prepared by removing essentially all the monoterpene fraction from Melaleuca alternifolia essential oil. The composition of the oil after removal of the monoterpene fraction is as follows:

| | |
|---|---|
| Terpen-4-ol | 52-54% |
| Alpha terpineol | 4-6% |
| Aromadendrene | 5-7% |
| Viridiflorene | 3-5% |
| Delta Cadinene | 1.5-3.5% |
| Globulol | 1-2% |
| Viridflorol | 0.3-0.6% |

The balance of the composition comprises a mixture of up to about 30 compounds in trace amounts. These compounds include other sesquiterpenes and higher molecular weight compounds.

Toxicity Studies

The acute oral toxicity of the above composition was investigated in 4 Sprague Dawley Specific Pathogen Free rats at doses of 500 and 1000 ppm at 10 mL/kg. The experimental procedure was based on OECD guidelines for the testing of chemicals No 401.

The test was administered orally once to 2 pairs of rats at the above doses. A third group was administered the vehicle only, solubilized vitamin E solution. The equivalent volumetric dose was 10 ml/kg for all groups.

Body weights were determined immediately before test item administration and at sacrifice on day 8. All animals were observed at frequent intervals on the day of test item administration and then daily for signs of toxicity over the 7 day experimental period, at the end of the experimental period, all animals were sacrificed and subjected to a gross necroscopy examination.

No mortalities were observed during the study.

No clinical abnormalities were observed for the duration of the study in any of the treated or control animals.

There were no gross abnormalities noted in the major organs of any animal at necroscopy.

Further analysis was carried out by gas chromatography testing of the kidneys and livers. No traces of the components of the above composition were found, indicating that all compounds are successfully excreted from the body.

Based on the results obtained from the study, the test composition, up to the highest doses tested 1000 ppm at 10 ml/kg did not produce toxicity in the Sprague Dawley rat, in the acute oral sighting study.

These results may be compared to tea tree oil, which has been reported to have an LD₅₀ of 1.9-2.6 ml/kg.

Microbiological Testing

Plates were prepared using Tryptic Soy Agar (bacteria) and Malt Extract (yeast and mould).

All micro-organisms were tested against ^{108(MacFarland Standard)} concentration of micro organism etc.

The results are shown in the following Table:

| | No. of tests | Average zone of |
|---|---|---|
| | carried | inhibition/mm |
| E. Coli | 105 | 24 |
| S. aureus | 68 | 30 |
| P. aeruginosa | 48 | 22 |
| C albicans | 60 | 35 |
| S epidermidis | 30 | 38 |
| Penicillium ssp | 68 | ng |
| Cladosporium | 89 | ng |
| A. Niger | 65 | ng |
| A. fumigatus | 5 | ng |
| P. Expansum | 5 | ng |
| S. chartarum | 4 | ng |
| Alternaria | 10 | ng |
| Aspergillus | 5 | ng |
| Fusarium | 4 | ng |
| B. subtilis | 15 | 35 |
| B. cereus | 20 | 35 |
| C. perfringens | 5 | ng |
| K. pneumoniae | 2 | ng |
| L. lactis | 1 | ng |
| M. Smegmatis | 5 | ng |
| S. Macescens | 3 | ng |
| S. pyogenes | 3 | ng |
| A. viridans | 10 | ng |
| E. aerogenes | 2 | ng |
| S. liquefaciens | 2 | ng |
| P. vulgaris | 15 | ng |
| S. enteridis | 4 | ng |
| P. mirabilis | 5 | ng |
| S. abaetetuba | 15 | ng |
| L. monocytogenes | 20 | ng |
| N. Gonorrhoeae | 5 | ng |
| Legionella | 10 | 35 |
| M. Gordanoae | 14 | 35 |
| M. catarrhalis | 3 | ng |

ng denotes no growth

Antiviral Testing

The virucidal efficacy of the composition of Example 1 against Coronavirus was tested. The results showed the composition to have an antiviral effect when diluted to 0.25% v/v.

Antiseptic Spray

The composition of Example 1 was formulated into a Nitrogen aerosol.

Gangrene

The aerosol formulation was sprayed immediately after debriding and directly onto the wound of an amputated gangrenous limb part. 11 patients have been treated according to this protocol. No re-infection was observed.

Palliative Care—Infected Cancer Tumours

The above aerosol spray has been observed to be effective in controlling infection and completely eradicating offensive odours caused by infected cancer tumours.

Tuberculosis

A trial of 3 patients infected with an antibiotic resistant strain of tuberculosis was conducted. Pulmonary administration of the antiseptic spray formulated to disperse particles of about 10 microns cleared the infection within 10 days of commencing treatment.

Periodontal Applications

The composition of the invention has been observed to successfully control intransigent periodontal infection in 5 patients.

Surgical Masks

The present inventor has observed that surgical masks typically only filter micro-organisms for a period of about 20 minutes after which they become ineffective. After this time, there is very little filtering, if any, of particles including micro-organisms. A mask was sprayed with an aerosol containing the composition from Example1 in medical grade alcohol as a solvent. The aerosol used nitrogen as the propellent and the particle size of the dispersed droplets was about 10 micron. The mask was sprayed at a rate of ½ g per second on both sides. The concentration of active compounds in the spray was 6000 ppm.

The sprayed mask was observed to increase the effective lifetime of the surgical mask from 20 minutes until up to about 3½ hours.

### Example 2

A second composition of the present invention was prepared from Melaleuca alternifolia as per Example 1. The composition of the oil is as follows:

| | |
|---|---|
| Terpen-4-ol | 60-62% |
| Alpha terpineol | 4.5-6.5% |
| Aromadendrene | 2.0-4.0% |
| Viridiflorene | 1.5-3.0% |
| Delta Cadinene | 1.5-3.5% |
| Globulol | 0.5-2.0% |
| Viridflorol | 0.3-0.8% |

Dermal Toxicity Studies

The composition of Example 2 was administered in a single bolus dermally to Guinea pigs in a vitamin E vehicle in doses of 5%, 15% and 25%.

The overall finding of the study was that the inventive composition, when administered as a single bolus to guinea pigs on an acute toxicity test by dermal application, did not demonstrate any increasing toxic effect corresponding to dosage. The absence of toxicity even at the highest test dosage of a 25% solution is suggestive that the anticipated human exposure to a 5% solution would have no adverse effect.

This result may be compared to commercially available melaleuca oil where contact dermatitis is recognized as a potential adverse side affect.

Pharmacokinetic Study
Results to be provided

Microbiological Testing

1. Methicillin-Resistant Staphylococcus aureus

A study was conducted to test a solution of the composition of Example 2 on a Methicillin-resistant strain of Staphylococcus aureus using the agar dilution technique. In the agar dilution technique, antimicrobial agents are incorporated into an agar medium. A diluted suspension of the test organism is then inoculated onto the medium, If the test organism is sensitive to the antibiotic n?? the agar medium, growth will be inhibited. If the organism grows on the anti-biotic containing medium, then it is resistant to this particular antibiotic.

Test Organisms

24 non-replicate isolates from individual patients of Methicillin resistant Staphylococcus aureus ATCC 33591 (mMRSA) were tested.

Plate Preparation

A 10% water soluble stock solution of the composition of Example 2 was added to sterile Mueller Hinton (MH) agar as follows:
0.125% 0.25 ml stock solution to 20 ml MH agar
0.25% 0.5 ml stock solution to 20 ml MH agar
0.5% 1 ml stock solution to 20 ml MH agar
1% 2 ml stock solution to 20 ml MH agar
2% 4 ml stock solution to 20 ml MH agar

The results are shown in the following Table:

| Well No. | Control | 0.125% | 0.25% | 0.5% | 1% | 2% |
|---|---|---|---|---|---|---|
| 1 | G | G | NG | NG | NG | NG |
| 2 | G | G | NG | NG | NG | NG |
| 3 | G | NG | NG | NG | NG | NG |
| 4 | G | G | NG | NG | NG | NG |
| 5 | G | G | NG | NG | NG | NG |
| 6 | G | G | NG | NG | NG | NG |
| 6 | G | G | NG | NG | NG | NG |
| 7 | G | G | NG | NG | NG | NG |
| 8 | G | NG | NG | NG | NG | NG |
| 9 | G | G | NG | NG | NG | NG |
| 10 | G | NG | NG | NG | NG | NG |
| 11 | G | NG | NG | NG | NG | NG |
| 12 | G | NG | NG | NG | NG | NG |
| 13 | G | NG | NG | NG | NG | NG |
| 14 | G | NG | NG | NG | NG | NG |
| 15 | G | NG | NG | NG | NG | NG |
| 16 | G | NG | NG | NG | NG | NG |
| 17 | G | G | NG | NG | NG | NG |
| 18 | G | NG | NG | NG | NG | NG |
| 19 | G | G | NG | NG | NG | NG |
| 20 | G | NG | NG | NG | NG | NG |
| 21 | G | NG | NG | NG | NG | NG |
| 22 | G | NG | NG | NG | NG | NG |
| 23 | G | G | NG | NG | NG | NG |
| 24 | G | NG | NG | NG | NG | NG |
| 25 | G | G | NG | NG | NG | NG |
| 26 | G | SLG | NG | NG | NG | NG |

Well No. 1-24 contain clinical isolates of mMRSA
Well No. 25 contains ATCC 33591 as a control
Well No. 26 contains ATCC 25923 as a control
G indicates growth or organisms
SG indicates scant growth of organisms
NG indicates no growth of organisms

The results show that all clinical isolates and ATCC control strains of

Staphylococcus aureus were inhibited at a concentration of 0.25% v/v.

2. Vancomycin resistant Enterococcus species 13 vancomycin strains of Enterococcus faecium and 7 vancomycin resistant strains of Enterococcus faecalis were tested against a composition as described in Example 2. Organisms were selected from a time period spanning 5 years to minimize the potential for testing of clonal isolates. 14 strains exhibit the vanB genotype (high level vancomycin resistance plus teicoplanin susceptibility) were tested compared to 6 strains with the vanA genotype (high level vancomycin and teicoplanin resistance). This distribution reflects the predominance of the vanB Enterococcus faecium in nosocomial outbreaks in Australia.

A composition according to Example 2 in a 10% water soluble solution was tested using the agar dilution technique as described above. The composition was tested at dilutions in agar of 0.125%, 0.25%, 0.5%, 1%, 2% and 4%.

The results are shown in the following Table:

| Well no. | Control | 0.125% | 0.25% | 0.5% | 1% | 2% | 4% |
|---|---|---|---|---|---|---|---|
| 1 | G | G | NG | NG | NG | NG | NG |
| 2 | G | G | G | NG | NG | NG | NG |
| 3 | G | G | NG | NG | NG | NG | NG |
| 4 | G | G | NG | NG | NG | NG | NG |
| 5 | G | G | NG | NG | NG | NG | NG |
| 6 | G | G | G | NG | NG | NG | NG |
| 7 | G | G | G | NG | NG | NG | NG |
| 8 | G | G | G | NG | NG | NG | NG |
| 9 | G | G | G | NG | NG | NG | NG |
| 10 | G | G | G | NG | NG | NG | NG |
| 11 | G | G | G | NG | NG | NG | NG |
| 12 | G | G | NG | NG | NG | NG | NG |
| 13 | G | G | G | NG | NG | NG | NG |
| 14 | G | G | G | NG | NG | NG | NG |
| 15 | G | G | G | NG | NG | NG | NG |
| 16 | G | G | G | NG | NG | NG | NG |
| 17 | G | G | G | NG | NG | NG | NG |
| 18 | G | G | G | NG | NG | NG | NG |
| 19 | G | G | G | NG | NG | NG | NG |
| 20 | G | G | G | NG | NG | NG | NG |
| 21 | G | G | G | NG | NG | NG | NG |
| 22 | G | G | G | NG | NG | NG | NG |

Wells 1 to 14 contain strains of Acinetobacter baumanni.
Well 15 contains Escherichia coli.
Wells 16 to 19 contain strains of Klebseiella pneumonia.
Well 20 contains Enterobacter cloacae.
Well 21 contains ATCC 25923 as a control.

The results show that all clinical isolates and ATT control strains of vancomycin resistant Enterococcus were inhibited at a concentration of 1% v/v irrespective of genotype and species.

3. Clinical Isolates of ESBL and MRA

Clinical isolates of Extended Spectrum Beta-Lactamase (ESBL) producing Gram Negative organisms and multi-resistant Acinetobacter baumanni (MRA) were tested against a 10% water soluble solution of the composition of Example 2 using the agar dilution technique as described above.

28 isolates with ESBL and 14 strains of MRA were tested. Organisms were selected from a time period spanning 3 years to minimize the potential for testing of clonal isolates. A selection of different species with ESBLs was tested including Escherichia coli.

A composition according to Example 2 in a 10% water soluble solution was tested using the agar dilution technique as described above. The composition was tested at dilutions in agar of 0.125%, 0.25%, 0.5%, 1%, 2% and 4%.

The results are shown in the following Table:

| Well No. | Control | 0.125% | 0.25% | 0.5% | 1% | 2% | 4% |
|---|---|---|---|---|---|---|---|
| 1 | G | G | G | NG | NG | NG | NG |
| 2 | G | G | G | NG | NG | NG | NG |
| 3 | G | G | G | NG | NG | NG | NG |
| 4 | G | G | G | NG | NG | NG | NG |
| 5 | G | G | G | NG | NG | NG | NG |
| 6 | G | G | G | NG | NG | NG | NG |
| 7 | G | G | G | NG | NG | NG | NG |
| 8 | G | G | G | NG | NG | NG | NG |
| 9 | G | G | G | NG | NG | NG | NG |
| 10 | G | G | G | NG | NG | NG | NG |
| 11 | G | G | G | NG | NG | NG | NG |
| 12 | G | G | NG | NG | NG | NG | NG |
| 13 | G | G | G | NG | NG | NG | NG |
| 14 | G | G | G | NG | NG | NG | NG |
| 15 | G | G | G | NG | NG | NG | NG |
| 16 | G | G | G | NG | NG | NG | NG |
| 17 | G | G | NG | NG | NG | NG | NG |
| 18 | G | G | G | NG | NG | NG | NG |
| 19 | G | G | G | NG | NG | NG | NG |
| 20 | G | G | G | NG | NG | NG | NG |
| 21 | G | G | G | NG | NG | NG | NG |
| 22 | G | G | G | NG | NG | NG | NG |

Well Nos. 1 to 4, 11, 12, 16, 17, 19 and 21 contain Escherichia coli.
Well Nos. 5, 13, 1518 and 20 contain Klebsiella pneumoniae
Well Nos. 6-8, 16 and 22 contain Enterobacter cloacae.

The results show that complete inhibition of all clinical isolates with ESBL and Acinetobacter baumanni was achieved at a concentration of the inventive composition of 0.5% v/v.

4. Anti-Tuberculosis Activity

Mycobacterium tuberculosis ATCC 27294 and Mycobacterium smegmatis ATCC 14468 was tested with a composition as described in Example 2.

Inocula Preparation

Loopfuls of cells from working cultures on Middlebrook 7H11 agar were transferred into 15 ml of Middlebrook 7H9 broth and then placed in a 100 ml flask with glass beads. The flask was shaken for 3 min using a mechanical-shaker. The suspensions were aspirated from the glass beads and transferred into another tube. The number of cells in suspensions was adjusted to ca0.5McFarland standard turbidity with 7H9. The working suspensions were prepared in a 1:5 dilution with saline and used within 2 h.

Modified EN 1276 Method

The European Standard EN 1276 modified to overcome potential problems with loss of activity dues to volatility of actives in the composition was used to evaluate various dilutions of the composition of Example 2.

Three ml of various concentrations of the composition of Example 2 were mixed with 3 ml of bacterial suspensions and left for various times of exposure (5-60 min). After exposure, 1 ml aliquots of the mixture were taken out and added to a tube containing 8 ml of neutralizer and 1 ml of SDW and mixed. After 5 min neutralization time, a 0.5 aliquot was removed for inoculation into a MGIT tube. This was placed on the Bactec 960 and monitored for growth of the mycobacterium for 4 weeks. For positive controls, the composition was replaced with saline and exposed for 60 minutes. For negative controls, a solution of the 10% composition (2%) which in a preliminary trial inhibited M. tuberculosus ATCC 27294 and M. smegmatis ATCC 14468, was mixed with bacterial suspensions and left for 60 min.

Test Composition

A solution of Example 2 at 10% containing 1% vitamin E was prepared using distilled water to concentrations between 0.03-4%.

The results are shown in the following Tables, which show the time to positive at various concentrations of a 10% solution of Example 2:

M. tuberculosis

| Exposure | | | | | | | | | |
|---|---|---|---|---|---|---|---|---|---|
| time | 0.03 | 0.06 | 0.125 | 0.25 | 0.5 | 1.0 | 2.0 | +ve | −ve |
| (min) | (h) | (h) | (h) | (h) | (h) | (h) | (h) | (h) | (h) |
| 5 | 340 | 334 | 366 | NG | NG | | | | |
| 15 | 381 | 357 | 398 | NG | NG | | | | |
| 30 | 419 | 380 | 424 | NG | NG | | | | |
| 60 | 314 | 443 | NG | NG | NG | | | 366 | NG |

M.smegmatis

| Exposure | | | | | | | | | |
|---|---|---|---|---|---|---|---|---|---|
| time | 0.03 | 0.06 | 0.125 | 0.25 | 0.5 | 1.0 | 2.0 | +ve | −ve |
| (min) | (h) | (h) | (h) | (h) | (h) | (h) | (h) | (h) | (h) |
| 5 | | | 63 | 62 | 75 | 101 | | | |
| 15 | | | 58 | 67 | 87 | NG | | | |
| 30 | | | 62 | 67 | 95 | NG | | | |
| 60 | | | 63 | 67 | 136 | NG | | 60 | NG |

The results show that the MIC for the composition of Example 2 was 0.125% after bacterial cells were exposed to the composition for 60 minutes using the BACTEC Mycobacteria Growth Indicator Tube system (BACTEC 960/MGIT). The result was validated using the rapid grower M. smegmatis. For M. smegmatis, the MIC for a 10% solution of the composition of Example 2 was 1% after the cells were exposed for 15 minutes. The results indicate that the Mycobacterium tuberculosis ATCC 27294 is susceptible to a solution of the inventive composition at a concentration of 0.125%

5. Avian Influenza

### H5N1 Avian Influenza Virus

This work was done to confirm the ability of the composition of Example 2 to act as a virucidal agent against a Vietnamese H5N1 highly pathogenic avian influenza virus strain. The trials were done in specific pathogen free chicken eggs, these being the most sensitive culture system for avian influenza viruses. The procedure was modified from ASTM E1052-96 “Standard Test Method for Efficacy of Antimicrobial Agents against Viruses in Suspension”.

Material and Methods

A 10% solution of the composition of Example 2 was made in dimethyl sulphoxide (DMSO) by adding 1.0 ml of the composition to 9.0 ml of DMSO and mixing thoroughly. A solution of 1% with no emulsifiers was used as supplied for virus treatment.

Virus

The virus used for this work was A/chicken/Vietname/8/2004H5N1 grown in the allantoic sac of embryonated, SPF chicken eggs. Infectious allantoic fluid was harvested, pooled and stored at −80° C. for this trial. The microstores pool reference number of this material is 0404-30-1550.

Trial 3-60, 120 and 240 Minute Contact Time with Virus Titrated in SPF Eggs

Virus Treatment

The 10% solution of Example 2 was diluted 1:5, 1:3.3 and 1:2.5 in phosphate buffered saline (pH 7.3) to give final concentrations of 2%, 3% and 4% respectively. Based on ASTM E1052-96, 0.1 ml of virus was mixed with 0.4 ml of the composition of Example 2 at concentrations of 2%, 3% and 4%. Mock-treated virus, consisting of 0.1 ml of virus mixed with 0.4 ml of a 1:5 dilution of DMSO without any inventive composition in PBS and untreated virus, consisting of 0.1 ml of virus mixed with 0.4 ml of PBS, were also prepared. All mixtures were incubated at room temperature for the respective times. 2%, 3% and 4% of the inventive composition were diluted 1:10 in PBS and 0.1 ml inoculated into 5 eggs each.

Virus Titrations

Trial 2—in SPF Eggs

Residual virus was assayed by making 10-fold dilutions in PBS of each virus mixture from 10⁻¹ to 10⁻⁸. 0.2 ml of virus mixture was added to 1.8 ml of PBS and mixed thoroughly to give a 10⁻¹ dilution. 0.2 ml of this was added to 1.8 ml of PBS and mixed thoroughly to give a 10⁻² dilution and so on to a final dilution of 10⁻⁸. 0.1 ml of each dilution was inoculated into the allantoic sac of embryonated, SPF chicken eggs, incubated at 37° C. for 3 days or until embryo death. Eggs were examined twice daily for viability. At death or after 3 days incubation all eggs were chilled overnight at 4° C. and then tested for the presence of haemagglutination and an indicator of virus infection. The residual infectivity titre was calculated by the method of Reed and Meunch.

Results

Eggs inoculated with the inventive composition alone showed no egg deaths, showing that mortalities were due to the effects of the virus inoculum.

Untreated Virus

| | Time post-inoculation | | | | | | |
|---|---|---|---|---|---|---|---|
| | 7/6 | | 8/6 | | 9/6 | | |
| Dilution | am | 7/6 pm | am | 8/6 pm | am | 9/6 pm | HA Result |
| 10⁻¹ | — | 5 | | | | | 5/5 positive |
| 10⁻² | — | — | 5 | | | | 5/5 positive |
| 10⁻³ | — | — | 5 | | | | 5/5 positive |
| 10⁻⁴ | — | — | 2 | 3 | | | 5/5 positive |
| 10⁻⁵ | — | — | — | 5 | | | 5/5 positive |
| 10⁻⁶ | — | — | — | 5 | — | — | 5/5 positive |
| 10⁻⁷ | — | — | — | 3 | — | — | 3/5 positive |
| 10⁻⁸ | — | — | — | — | — | — | 5/5 negative |

Virus titre = 10^{7.1} egg infectious doses per 0.1 ml

Mock-Treated Virus

| | Time post-inoculation | | | | | | |
|---|---|---|---|---|---|---|---|
| | 7/6 | | 8/6 | | 9/6 | | |
| Dilution | am | 7/6 pm | am | 8/6 pm | am | 9/6 pm | HA Result |
| 10⁻¹ | — | 5 | | | | | 5/5 positive |
| 10⁻² | — | — | 5 | | | | 5/5 positive |
| 10⁻³ | — | — | 5 | | | | 5/5 positive |
| 10⁻⁴ | — | — | 2 | 3 | | | 5/5 positive |
| 10⁻⁵ | — | — | 3 | 2 | | | 5/5 positive |
| 10⁻⁶ | 2 | — | 3 | — | | | 5/5 positive |
| 10⁻⁷ | — | — | 4 | — | — | — | 4/5 positive |
| 10⁻⁸ | — | — | 1 | — | — | — | 1/5 positive |

Virus titre = 10^{7.5} egg infectious doses per 0.1 ml

2% MegaBac™ Treated Virus

60 Minute Contact Time

| | Time post-inoculation | | | | | | |
|---|---|---|---|---|---|---|---|
| | 2/8 | | 3/8 | | 4/8 | | |
| Dilution | am | 2/8 pm | am | 3/8 pm | am | 4/8 pm | HA Result |
| 10⁻¹ | — | 5 | | | | | 5/5 positive |
| 10⁻² | — | — | 5 | | | | 5/5 positive |
| 10⁻³ | — | — | 5 | | | | 5/5 positive |
| 10⁻⁴ | — | — | 3 | — | | | 3/5 positive |
| 10⁻⁵ | — | — | — | — | | | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 negative |

Virus titre = 10^{4.1} egg infectious doses per 0.1 ml

120 Minute Contact Time

| | Time post-inoculation | | | | | | |
|---|---|---|---|---|---|---|---|
| | 20/8 | | 21/8 | | 22/8 | | |
| Dilution | am | 20/8 pm | am | 21/8 pm | am | 22/8 pm | HA Result |
| 10⁻¹ | — | 5 | | | | | 5/5 positive |
| 10⁻² | — | — | 5 | | | | 5/5 positive |
| 10⁻³ | — | — | 5 | | | | 5/5 positive |
| 10⁻⁴ | — | — | 4 | — | — | — | 2/5 positive |
| 10⁻⁵ | — | — | 1 | — | — | — | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 positive |

Virus titre = 10^{4.5} egg infectious doses per 0.1 ml

240 Minute Contact Time

| | Time post-inoculation | | | | | | |
|---|---|---|---|---|---|---|---|
| | 27/8 | | 28/8 | | 29/8 | | |
| Dilution | am | 27/8 pm | am | 28/8 pm | am | 29/8 pm | HA Result |
| 10⁻¹ | — | 5 | | | | | 5/5 positive |
| 10⁻² | — | — | 5 | | | | 5/5 positive |
| 10⁻³ | — | — | 3 | — | — | — | 5/5 positive |
| 10⁻⁴ | — | — | — | — | — | | 3/5 positive |
| 10⁻⁵ | — | — | — | — | — | — | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 positive |

Virus titre = 10^{3.1} egg infectious doses per 0.1 ml

3% MegaBac™ Treated Virus

60 Minute Contact Time

| | Time post-inoculation | | | | | | |
|---|---|---|---|---|---|---|---|
| | 2/8 | 2/8 | 3/8 | | | | |
| Dilution | am | pm | am | 3/8 pm | 4/8 am | 4/8 pm | HA Result |
| 10⁻¹ | — | 5 | | | | | 5/5 positive |
| 10⁻² | — | — | 5 | | | | 5/5 positive |
| 10⁻³ | — | — | 2 | — | — | — | 2/5 positive |
| 10⁻⁴ | — | — | — | — | — | — | 0/5 positive |
| 10⁻⁵ | — | — | — | — | — | — | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 negative |

Virus titre = 10^{2.9} egg infectious doses per 0.1 ml

120 Minute Contact Time

| | Time post-inoculation | | | | | | |
|---|---|---|---|---|---|---|---|
| | 20/8 | 20/8 | 21/8 | | | | |
| Dilution | am | pm | am | 21/8 pm | 22/8 am | 22/8 pm | HA Result |
| 10⁻¹ | — | 5 | | | | | 5/5 positive |
| 10⁻² | — | — | 5 | | | | 5/5 positive |
| 10⁻³ | — | — | 4 | — | — | — | 5/5 positive |
| 10⁻⁴ | — | — | 1 | — | — | — | 1/5 positive |
| 10⁻⁵ | — | — | — | — | — | — | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 positive |

Virus titre = 10^{3.5} egg infectious doses per 0.1 ml

240 Minute Contact Time

| | Time post-inoculation | | | | | | |
|---|---|---|---|---|---|---|---|
| | 27/8 | 27/8 | 28/8 | | | | |
| Dilution | am | pm | am | 28/8 pm | 29/8 am | 29/8 pm | HA Result |
| 10⁻¹ | — | 5 | | | | | 5/5 positive |
| 10⁻² | — | — | 5 | | | | 5/5 positive |
| 10⁻³ | — | — | 2 | — | — | — | 2/5 positive |
| 10⁻⁴ | — | — | — | — | — | | 0/5 positive |
| 10⁻⁵ | — | — | — | — | — | — | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 positive |

Virus titre = 10^{2.9} egg infectious doses per 0.1 ml

4% Treated Virus

60 Minute Contact Time

| | Time post-inoculation | | | | | | |
|---|---|---|---|---|---|---|---|
| | 13/8 | 13/8 | 14/8 | | | | |
| Dilution | am | pm | am | 14/8 pm | 15/8 am | 15/8 pm | HA Result |
| 10⁻¹ | — | 5 | | | | | 5/5 positive |
| 10⁻² | — | 5 | | | | | 5/5 positive |
| 10⁻³ | — | 3 | — | — | — | — | 3/5 positive |
| 10⁻⁴ | — | — | — | — | — | — | 0/5 positive |
| 10⁻⁵ | — | — | — | — | | | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 negative |

Virus titre = 10^{3.1} egg infectious doses per 0.1 ml

120 Minute Contact Time

| | Time post-inoculation | | | | | | |
|---|---|---|---|---|---|---|---|
| | 20/8 | 20/8 | 21/8 | | | | |
| Dilution | am | pm | am | 21/8 pm | 22/8 am | 22/8 pm | HA Result |
| 10⁻¹ | — | 5 | | | | | 5/5 positive |
| 10⁻² | — | — | 5 | | | | 5/5 positive |
| 10⁻³ | — | — | 1 | — | — | — | 1/5 positive |
| 10⁻⁴ | — | — | — | — | — | — | 0/5 positive |
| 10⁻⁵ | — | — | — | — | — | — | 0/5 positive |
| 10⁻⁶ | — | — | — | — | — | — | 0/5 positive |
| 10⁻⁷ | — | — | — | — | — | — | 0/5 positive |
| 10⁻⁸ | — | — | — | — | — | — | 0/5 positive |

Virus titre = 10^{2.7} egg infectious doses per 0.1 ml

240 Minute Contact Time

| | Time post-inoculation | | | | | | |
|---|---|---|---|---|---|---|---|
| | 3/9 | 30/8 | | | | | |
| Dilution | am | pm | 4/8 am | 31/8 pm | 5/9 am | 5/9 pm | HA Result |
| 10⁻¹ | — | — | — | — | 1 | — | 0/5 positive |
| 10⁻² | — | — | — | — | — | — | 0/5 positive |
| 10⁻³ | — | — | — | — | — | — | 0/5 positive |
| 10⁻⁴ | — | — | — | — | — | — | 0/5 positive |
| 10⁻⁵ | — | — | — | — | — | — | 0/5 positive |
| 10⁻⁶ | | | | | | | |
| 10⁻⁷ | | | | | | | |
| 10⁻⁸ | | | | | | | |

Virus titre = 10 egg infectious doses per 0.1 ml

### Summary of Titration Results of Trial 3

Log 10 residual virus titre after treatment in (EID₅₀/0.1 ml)

| | Treatment Time | | |
|---|---|---|---|
| concentration | 60 Minutes | 120 Minutes | 240 Minutes |
| 2% | 4.1 | 4.5 | 3.1 |
| 3% | 2.9 | 3.5 | 2.9 |
| 4% | 3.1 | 2.7 | 0 |
| Untreated virus | 7.1 | | |
| Mock-treated virus | 7.5 | | |

Eggs inoculated with 1:10 dilutions of the inventive solutions alone showed no egg deaths, showing that mortalities were due to the effects of the virus inoculum.

It may be appreciated that the composition of the present invention exhibits negligible levels of toxicity when compared to Melaleuca essential oil which contains significant amounts of monoterpenes. This enables the composition to be used in a wide range of applications for which conventional Melaleuca oil would be unsuitable. A particular advantage of the reduction in toxicity is the ability to use the composition of the present invention in medical applications.

Despite the dramatic reduction in monoterpene content, the composition of the present invention has not only retained but improved its efficacy. It will be appreciated that various changes or modifications may be made to the invention as described and claimed herein without departing from the spirit and scope thereof.

1. A composition derived from an essential oil of a Melaleuca plant, wherein at least about 80% of the monoterpene content of the oil has been removed.

2. The composition of claim 1, wherein the Melaleuca plant is Melaleuca alternifolia.

3. The composition of claim 1, wherein the monoterpenes have been removed under reduced pressure at a temperature of less than 50° C.

4. The composition of claim 2, which comprises between about 50 to about 70% terpen-4-ol and between about 8 to about 30% sesquiterpenes.

5. The composition of claim 2, which comprises between about 60 to about 65% terpen-4-ol and between about 8 to about 25% sesquiterpenes.

6. The composition of claim 2, having the following composition;

| | |
|---|---|
| Terpen-4-ol | 52-62% |
| Alpha terpineol | 4-6.5% |
| Aromadendrene | 2-7% |
| Viridiflorene | 1.5-5% |
| Delta Cadinene | 1.5-3.5% |
| Globulol | 0.5-2% |
| Viridiflorol | 0.3-0.8% |

7. An antimicrobial composition derived from an essential oil of a Melaleuca plant, wherein at least about 80% of the monoterpene content of the oil has been removed.

8. The composition of claim 7, further comprising a liquid solvent, carrier and/or diluent.

9. A method of sanitizing a surface comprising applying to the surface an effective amount of the composition of claim 8.

10. An antimicrobial aerosol composition comprising the composition of claim 1 dispersed in a propellent.

11. The composition of claim 10, wherein the propellant comprises nitrogen.

12. A method for controlling airborne infectious agents in an atmospheric environment, the method comprising dispersing an effective amount of the aerosol of claim 8 into the environment.

13. The method of claim 12, wherein the infectious agent is a virus.

14. The method of claim 12, wherein the virus is an avian influenza virus.

15. A topical therapeutic composition comprising the composition of claim 1 and a topically acceptable carrier, diluent, excipient and/or adjuvant.

16. The composition of claim 15 for use as an analgesic.

17. The composition of claim 15 for use as an anti-inflammatory agent.

18. A pharmaceutical composition for treating or controlling an infection in an mammal, the composition comprising an effective amount of the composition of claim 1 together with a pharmaceutically acceptable carrier diluent, excipient and/or adjuvant.

19. The composition of claim 18, in the form of an aerosol.

20. A method for the treatment or prophylaxis of an infection in an mammal or bird, the method comprising administering to the mammal an effective amount of the pharmaceutical composition of claim 18.

21. A method for the treatment or prophylaxis of a respiratory infection in an mammal or bird, the method comprising administering to the mammal an effective amount of the pharmaceutical composition of claim 19.

22. The method of claim 21, wherein the infectious agent is Mycobacterium tuberculosis.

23. The method of claim 21, wherein the infectious agent is an influenza virus.

24. The method of claim 23, wherein the influenza virus is type A.

25. A fabric to which a composition of claim 1 has been applied.

26. A surgical face mask to which the composition of claim 1 has been applied.

27. A medical device treated with the composition of claim 1.
